(19) United States
(12) Patent Application Publication
(10) Pub. No.: US 2022/0288113 A1
(43) Pub. Date: Sep. 15, 2022

(54) ENHANCED NITRATE, ELEMENTAL METAL, AND AMINO ACID COMPOSITIONS AND METHODS OF USE

(71) Applicant: ThermoLife International, LLC, Signal Hill, CA (US)

(72) Inventors: Ronald Kramer, Signal Hill, CA (US); Alexandros Nikolaidis, Nea Kallikratia (GR)

(21) Appl. No.: 17/693,191

(22) Filed: Mar. 11, 2022

Related U.S. Application Data
(63) Continuation of application No. PCT/US2022/020054, filed on Mar. 11, 2022.
(60) Provisional application No. 63/159,990, filed on Mar. 11, 2021.

(51) Int. Cl. A61K 33/26; A61K 33/00; A61K 31/198; A61K 31/405; A61K 31/4172; A61K 33/08; A61K 33/24; A61K 31/375; A61K 36/21

(52) U.S. Cl. CPC A61K 33/26; A61K 33/00; A61K 31/198; A61K 31/405; A61K 31/4172; A61K 33/08; A61K 33/24; A61K 31/375; A61K 36/21

(57) ABSTRACT

The disclosure is directed to methods of at least reducing nitrosamine formation, preventing formation of, or even reversing formation of, nitrosamines, improving athletic performance, and increasing aerobic capacity, stamina, muscle strength, endurance, and/or time to exhaustion in a subject. The disclosure is also directed to related compositions including a nitrate (source of nitrate anion NO₃−) and/or a nitrite (source of nitrite anion NO₂−), an amine moiety containing compound, and an elemental metal. In other implementations, the compositions may also include an acid.

### CROSS REFERENCE TO RELATED APPLICATIONS

This application is a continuation of PCT/US22/20054, filed Mar. 11, 2022, which claims priority to U.S. Provisional Patent Application No. 63/159,990, filed on Mar. 11, 2021, the contents of each of which are incorporated herein by reference in their entirety.

### BACKGROUND

Formation of N-nitroso compounds is a multiple step process. In human nutrition orally consumed nitrate is converted partially into nitrite after consumption. Then the nitrite reacts with natural or synthetic organic amine compounds in food or water to form new combinations called N-nitroso compounds (either nitrosamines or nitrosamides).

N-Nitrosamines are formed in the nitrosation reaction from primary, secondary and tertiary amines as well tertiary ammonium compounds, such as choline and carnitine. Most nitrosamines are brightly colored, since the nitroso group is a strong chromatophore.

Many of these N-nitroso compounds have been found to be carcinogenic in all the animal species tested.

Nitrosamine formation by nitrite happens at a much greater ratio in acidic environments, such as those of the stomach. At least 75% of the 120 N-nitroso compounds have been found to be carcinogenic to animals.

There are other nitrosamines too, such as N-nitrosodimethylamine (MDMA), N-nitrosodiethylamine (NDEA), N-nitrosodibutylamine (NDBA), N-nitrosopiperidine (NPIP), N-nitrosopyrrolidine (NPYR), and N-nitrosothiazolidine (NTHZ), which people can be exposed to by any sources, such as contaminants in drinking water, contaminants in commercially sold drugs (the popular medicine Ranitidine was pulled off the market by the FDA in 2019).

Therefore, there have been and still exist concerns about the co-administration of nitrates and nitrites with amino acids.

### SUMMARY OF THE INVENTION

The disclosure relates to compositions, kits, and methods for reducing nitrosamine formation from an amine moiety containing compound.

A method of reducing nitrosamine formation from an amine moiety containing compound is disclosed that comprises co-administering the amine moiety containing compound with an elemental metal and a nitrate and/or a nitrite.

In some implementations, the amine moiety containing compound is an amino acid, for example, in a salt form. In other implementations, the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine. In some implementations, the elemental metal is selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

In some implementations, the method further comprises administering an acid, wherein the acid is co-administered with the amine moiety containing compound, the elemental metal, and the nitrate and/or the nitrite. In such implementations, nitrosamine formation of the amine moiety containing compound is prevented.

A method of reducing nitrosamine formation in a composition comprising an amine-moiety containing compound and a nitrosating agent is also disclosed. The method comprises adding an elemental metal to the composition.

In some implementations, the nitrosating agent is selected from the group consisting of: a nitrate, a nitrite, nitric oxide, and nitrogen dioxide. In particular implementations, the nitrosating agent is nitric oxide and/or nitrogen dioxide.

In some implementations, ingestion of the composition comprising the elemental metal forms a lesser amount of nitrosamine compared to ingestion of the composition without the elemental metal.

In some implementations, the elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

In some implementations, the amine moiety containing compound is an amino acid, for example, in a salt form, and the composition does not form nitrosamines upon ingestion.

In some implementations, the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine.

In certain implementations, the method further comprises adding an acid to the composition.

A composition with reduced nitrosamine formation is also disclosed. The composition comprises an elemental metal, a nitrate and/or a nitrite, and an amine moiety containing compound.

In some implementations, the composition further comprises an acid.

In some implementations, the elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

In some implementations, the amine moiety containing compound is an amino acid, for example, an amino acid in a salt form. In other implementations, the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine.

### DETAILED DESCRIPTION

Detailed aspects and applications of the disclosure are described below in the following detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant art, that implementations of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “about” refers to a deviation of no more than 5% of the given value, for example a deviation of 3%, 2%, 1%, 0.5%, or 0.1% of the given value.

As used herein, the term “acceptable” is a phrase used in its broadest sense and may describe ingredients of a composition that meet Food and Drug Administration (FDA) standards, United States Pharmacopeia (USP) standards, U.S. Department of Agriculture (USDA) standards for food-grade materials, commonly accepted standards of the nutritional supplement industry, industry standards, botanical standards, or standards established by any individual. These standards may delineate acceptable ranges of aspects of ingredients of a composition such as edibility, toxicity, pharmacological effect, or any other aspect of a chemical, composition, or preparation used in implementations of a composition.

As used herein, “amino acid” is a term used in its broadest sense and may refer to an amino acid in its many different chemical forms, including a single administration amino acid in its free form, such as L-cysteine, its physiologically active salts (such as the nitrate salts), its combinations with its various salts, its tautomeric, and/or isomeric forms, its ester and amide forms, and/or its decarboxylation products. Amino acids include, by way of non-limiting example: agmatine, alanine, beta alanine, arginine, asparagine, aspartic acid, cysteine, glutamine, glutamic acid, glycine, histidine, isoleucine, leucine, lysine, methionine, phenyl-beta-alanine, phenylalanine, proline, serine, threonine, tryptophan, tyrosine, and valine.

As used herein, the term “amine moiety containing compound” refers to a compound that contains a functional group that contains a basic nitrogen atom with a lone pair, for example, a primary amine group (-NH₂-R, also known as an amino group), a secondary amine group (-NH-R¹-R²), or a tertiary amine group (-N-R¹-R²-R³). Accordingly, an amine moiety containing compound includes the essential amino acids as well as gamma-aminobutyric acid (GABA).

As used herein, the term “composition” refers to both a mixture of ingredients or constituents as well as a combination of capsules that contains different ingredients or constituents. Accordingly, in certain implementations, a composition encompasses separate capsules that are packaged together and are meant to be taken together.

As used herein, the term “elemental metal” refers to the neutral-charged state of a metal element, in other words, a metal in its uncharged elemental form and not in a salt form or charged form (exemplary salt forms and charged forms include the oxide, hydroxide, carbonate, chloride, lactate, citrate, aspartate, glycinate, and gluconate of the metal). As such, as used herein, elemental metals and salts of the same metals are different constituents. A description that a composition comprises an elemental metal cannot be satisfied by the presence of a metal salt, and vice versa. For example, a composition that consists of magnesium citrate is not a composition that comprises elemental magnesium in spite of any description that magnesium citrate provides some amount of elemental magnesium.

The elemental metals described herein include elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, elemental nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, elemental manganese, and elemental iron.

As used herein, “pump” refers to the feeling of better blood flow as described by athletes, which can be verified by user experience as well as scientific methods, such as measurement of muscle circumference after exercise, doppler ultrasound measurement of blood flow, measurement of vein size with a vein viewer, and the like.

As used herein, the term “nitrosating agent” refers to an agent that causes nitrosation of another compound. For example, a nitrosating agent includes, but is not limited to, a source of nitrite anion (NO₂−), a source of nitrate anion (NO₃−), nitric oxide (NO), and nitrogen dioxide (NO₂).

The inventors have had commercial success in their amino acid with nitrate or nitrite inventions due to health and other benefits of nitrate supplementation (for example, reduction in blood pressure, improvement in endothelial function, and increased exercise performance). For the nitrite product, there still remains dosing restrictions to ensure safety compared to nitrate, and so there is need for further improvements.

Disclosed herein are new compositions that have reduced risk for nitrosamine formation that comprise a nitrate (source of nitrate anion NO₃−) and/or a nitrite (source of nitrite anion NO₂−), an amine moiety containing compound, and an elemental metal. In some implementations, the amine moiety containing compound is an amino acid. In other implementations, the amine moiety containing compound is selected from the group consisting of agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine. In other implementations, the compositions may also include an acid.

In some implementations, the nitrate and/or nitrite is a nitrate salt and/or nitrite salt of an amino acid or amino acid derivative (for example, citrulline nitrate, citrulline nitrite, betaine nitrate, betaine nitrite), an inorganic nitrate salt (for example, magnesium nitrate, sodium nitrate, potassium nitrate, calcium nitrate, lithium nitrate, magnesium nitrite, sodium nitrite, potassium nitrite, calcium nitrite, and lithium nitrite or their mixed salts, co-crystalline formulation and hydrates), or a natural source. For natural sources, the nitrate and/or nitrite has been concentrated and/or isolated from a natural source, such as a botanical nitrate source or a botanical nitrite source. Examples of natural nitrate and/or nitrite sources include, but are not limited to, beet juice, beet juice powder, concentrated beet juice powder, celery powder, spinach and red spinach extract, and Amaranthus extract. In particular implementations, the nitrate and/or nitrite content of natural nitrate and/or nitrite sources is standardized to provide the sufficient amount of nitrate. In some implementations, the composition comprises more than one source or form of nitrate and/or nitrite.

In some implementations, elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

In particular implementations, the composition comprises elemental iron as the elemental metal component, beta alanine and proline as amine moiety containing compound components, and citrulline nitrate and betaine nitrate as nitrate and/or nitrite components. In some implementations, the composition further comprises citrulline as a further amine moiety containing compound component, and citrulline nitrate as a further nitrate and/or nitrite component. In particular implementations, the composition further comprises at least one antioxidant, for example the composition may further comprise black tea leaf extract, ferulic acid, and L-theanine. In some implementations, the composition further comprises caffeine. The composition may also comprise calcium and folate.

In other particular implementations, the composition comprises elemental iron as the elemental metal component; cysteine as the amine moiety containing compound component; and an Amaranthus extract as the nitrate and/or nitrite components. In some implementations, the composition further comprises an acid, for example ascorbic acid (vitamin C). In particular implementations, the composition further comprises at least one antioxidant. For example the composition may further comprise theanine. In some implementations, the composition may also comprise calcium and folate/5-MTHF and/or potassium molybdate.

It was also surprisingly discovered that co-administration of a nitrate (source of nitrate anion NO₃−) and/or nitrite (source of nitrite anion NO₂−) with other elemental metals (for example, elemental magnesium and elemental zinc) also increased the effectiveness of the nitrate and/or nitrite. Thus, the present disclosure is also directed to methods that increase the effectiveness of the nitrate anion (NO₃−) and/or nitrite anion (NO₂−) in providing a beneficial effect to a subject. For example, the methods may improve athletic performance in a subject. In some aspects of the methods of improving athletic performance in a subject, the aerobic capacity, stamina, muscle strength, endurance, and/or time to exhaustion in a subject are/is increased.

Physiological effects from the ingestion of the disclosed compositions may occur within 15 minutes and as early as 5 minutes after ingestion of the composition. This time frame coincides with when pre-workout supplements are typically administered. While it is recommended that most pre-workout supplements should be taken around 30 minutes prior to training, users often take their supplements just before training. Thus, the disclosed composition is more compatible with the habits of athletes and other users of pre-workout supplements. Instead of needing to remember to take their nitrate supplements three hours before training in order to maximize the benefits of nitrate supplementation, they only need to ingest the claimed composition a few minutes, for example five minutes, before training. The minimal delay between ingestion of the claimed composition and physiological effects attributed to inorganic nitrates can easily be satisfied by the warmup period at the start of training. Accordingly, in certain implementations of the methods of improving athletic performance or of increasing stamina, muscle strength, endurance, and/or time to exhaustion, the subject is co-administered an elemental metal and a nitrate (source of nitrate anion NO−) and/or a nitrite (source of nitrite anion NO₂−) within 30 minutes prior to the session of physical activity or training.

Because of the rapid and potent action of the disclosed compositions, these compositions can be used for all situations where nitrate's effects are beneficial and a novel, rapid effect is desired.

The elemental metal is an alkaline earth metal, an alkali metal, or a transition metal. In some implementations, the elemental metal may be elemental magnesium, elemental calcium, elemental lithium, elemental zinc, elemental iron, and the like. In some implementations, the nitrate (source of nitrate anion NO₃−) may be a salt of nitric acid, for example potassium nitrate, sodium nitrate, magnesium nitrate, proline nitrate, betaine nitrate, beta-alanine nitrate, citrulline nitrate, and the like. In other aspects, the nitrate anion is a botanical nitrate source. In other implementations, the composition may further include an acid. In certain implementations, the acid is a liquid acid, such as hydrochloric acid, phosphoric acid, nitric acid, or sulfuric acid. In other implementations, a solid acid is used, such as citric acid which is dissolved in water. The acid can be dissolved in water before adding the elemental metal and nitrate.

In some implementations, the composition comprises an effective amount of the nitrate (source of nitrate anion NO₃−) and/or nitrite (source of nitrite anion NO₂−) and an effective amount of the elemental metal, wherein the effective amount of the elemental metal supports/enhances the effectiveness of the nitrate anion (NO₃−) in providing a beneficial effect. Accordingly, in some implementations, the effective amount of the nitrate (source of nitrate anion NO₃−) and/or nitrite (source of nitrite anion NO₂−) is an amount sufficient to cause vasodilation, to increase athletic performance, and the like in a subject. In other implementations, the effective amount of the nitrate (source of nitrate anion NO₃−) and/or nitrite (source of nitrite anion NO₂−) in the composition is less than the amount of nitrate anion (NO₃−) needed alone to cause vasodilation or to increase athletic performance in a subject, while the effective amount of the elemental metal is an amount sufficient to support the nitrate (source of nitrate anion NO₃−) and/or nitrite (source of nitrite anion(NO₂−)) in causing vasodilation or increasing athletic performance in a subject.

In certain implementations, the composition comprises about 1 to about 900 mg elemental magnesium and a nitrate (source of nitrate anion NO₃−) and/or a nitrite (source of nitrite anion NO₂−), wherein the source of inorganic nitrate provides about 5 to about 2000 mg of nitrate anion (NO₃−). In some aspects, the nitrate (source of nitrate anion NO₃−) and/or the nitrite (source of nitrite anion NO₂−) provides about 30 mg to 2000 mg of nitrate anion (NO₃−), about 50 mg to about 2000 mg of nitrate anion (NO₃−), about 5 mg to about 1000 mg of nitrate anion (NO₃−), about 30 mg to about 1000 mg of nitrate anion (NO₃−), about 50 mg to about 1000 mg of nitrate anion (NO₃−), about 5 mg to about 600 mg of nitrate anion (NO₃−), about 30 mg to about 600 mg of nitrate anion (NO₃−), about 50 mg to about 600 mg of nitrate anion (NO₃−), about 5 mg to about 500 mg of nitrate anion (NO₃−), about 30 mg to about 500 mg of nitrate anion (NO₃−), or about 50 mg to about 500 mg of nitrate anion (NO₃−). In certain implementations, the nitrate (source of nitrate anion NO₃−) and/or the nitrite (source of nitrite anion NO₂−) is a salt of nitric acid, for example, magnesium nitrate or proline nitrate.

In some implementations, the molar ratio of the nitrate (source of nitrate anion NO₃−) and/or nitrite (source of nitrite anion NO₂−) to the elemental metal in the composition is at least 2:1. The doses of the elemental metal and the nitrate (source of nitrate anion NO₃−) and/or the nitrite (source of nitrite anion NO₂−) can be adjusted according to the subject's weight, age, and health status. Typically, normotensive subjects require less of elemental metal and nitrate anion (NO₃−) than hypertensive subjects, and hypotensive subjects will require even less elemental metal and nitrate anion (NO₃−) than normotensive subjects. In some implementations, the composition comprises 1-5000 mg NO₃− and/or NO₂− and 1-10000 mg elemental metal. In other implementations, the composition comprises 5-200 mg NO₃− and/or NO₂− and 5-1000 mg elemental metal. In some implementations, the amount of elemental metal in the composition is between 1 mg and 800 mg or between 5 mg and 400 mg. In some implementations, the amount of the nitrate (source of nitrate anion NO₃−) and/or the nitrite (source of nitrite anion NO₂−) is between 30 mg and 2000 mg or between 50 mg and 600 mg.

In some implementations, the composition is in the form of a capsule, a cachet, a pill, a tablet, a powder, a granule, a pellet, a bead, a particle, a troche, a lozenge, a pastille, a solution, an elixir, a syrup, a tincture, a suspension, an emulsion, a mouthwash, a spray, a drop, an ointment, a cream, a gel, a paste, a transdermal patch, a suppository, a pessary, cream, a gel, a paste, a foam, and combinations thereof. The composition may further comprise an acceptable additive and/or an acceptable carrier. The acceptable additive may be selected from at least one member from the group consisting of: a solubilizer, an enzyme inhibiting agent, an anticoagulant, an antifoaming agent, an antioxidant, a coloring agent, a coolant, a cryoprotectant, a hydrogen bonding agent, a flavoring agent, a plasticizer, a preservative, a sweetener, and a thickener. The acceptable carrier may be selected from at least one member from the group consisting of: an excipient, a lubricant, a binder, a disintegrator, a diluent, an extender, a solvent, a suspending agent, a dissolution aid, an isotonization agent, a buffering agent, a soothing agent, and an amphipathic lipid delivery system. In some implementations, the composition is in a form suitable for oral administration. In other implementations, the composition is in a form suitable for inhalation of the gases produced when in contact with an acidified solvent.

The elemental metal in the composition may be in any form, for example, a powder or granules. In certain implementations of the composition, the nitrate and/or the nitrite is packaged in a capsule, a cachet, a pill, a tablet, a powder, a granule, a pellet, a bead, a particle, a troche, or a pastille; and the elemental metal is packaged in a capsule, a cachet, a pill, a tablet, a powder, a granule, a pellet, a bead, a particle, a troche, or a pastille. In another implementations, the nitrate and/or the nitrite is packaged in a capsule, a cachet, a pill, a tablet, a powder, a granule, a pellet, a bead, a particle, a troche, or a pastille; the elemental metal is packaged in a capsule, a cachet, a pill, a tablet, a powder, a granule, a pellet, a bead, a particle, a troche, or a pastille; and the acid is packaged separately from the nitrate and/or nitrite and the elemental metal. In some implementations, the nitrate and/or the nitrite, the elemental metal, and the acid are in solid forms. In a particular implementation, the nitrate and/or nitrite and the elemental metal are packaged together, for example, in one capsule, one cachet, one pill, or one tablet.

In still other implementations, the composition comprises a source of nitrite anion (NO₂−) instead of a source of nitrate anion (NO₃−). For example, the composition comprises a source of nitrite anion (NO₂−), an amino acid, and an elemental metal. In some implementations, the composition comprises a source of nitrite anion, an amino acid, an elemental metal, and an acid.

In some implementations, the elemental metal in the composition is covered or microencapsulated with a suitable material that is poorly soluble in water but soluble in the acidic environment of the stomach, for example, magnesium oxide, cellulose polymers, alginates (such as calcium alginate), or aluminum hydroxide.

In some implementations, proline nitrate is the nitrate (source of nitrate anion NO₃−) and/or nitrite (source of nitrite anion NO₂−) in the composition. In some implementations, the magnesium nitrate is the nitrate (source of nitrate anion NO₃−) and/or the nitrite (source of nitrite anion NO₂−) in the composition. In such implementations, the magnesium nitrate may be anhydrous or hydrated. The degree of hydration of the magnesium nitrate is between one and six molecules of water per molecule of magnesium nitrate. In a particular implementation, magnesium nitrate hexahydrate is the salt of nitric acid in the composition.

In some implementations, the disclosed composition further comprises an acid. The acid is added to ensure the pH of the stomach upon ingestion of the claimed composition remains acidic. The acid component can be any acid suitable for human consumption, for example, citric acid, succinic acid, malic acid, ascorbic acid, or tartaric acid. In some implementations, the acid is in a solid form, for example, a powder. Thus, the amount of the acid in the composition in some implementations is between 50 mg and 20,000 mg, between 50 mg and 2000 mg, between 50 mg and 1000 mg, between 100 mg and 20,000 mg, between 100 mg and 2000 mg, between 100 mg and 1000 mg, between 200 mg and 20,000 mg, between 200 mg and 2000 mg, between 200 mg and 1000 mg, between 300 mg and 20,000 mg, between 300 mg and 2000 mg, between 300 mg and 1000 mg, between 500 mg and 20,000 mg, between 500 mg and 2000 mg, or between 500 mg and 1000 mg. In some implementations, the acid component of the composition is a vinegar. In some implementations, the disclosed composition does not comprise an acid, but the composition is administered with an acid. For example, the composition is co-administered with an acidic solution with pH between 2-6, for example, diluted vinegar or a solution of citric acid. In some implementations, the acidic solution is diluted acetic acid, nitric acid, sulfuric acid, and the like.

The disclosed composition may be in the form of a capsule, tablet, pill, liquid, liquid suspension, vapor, powder, granulate, pulverulence, or a combination thereof. In one implementation, the disclosed composition is in a solid form. In some implementations, the elemental metal and the nitrate (source of nitrate anion NO₃−) and/or the nitrite (source of nitrite anion NO₂−), and in some implementations the acid and/or amino acid, are combined into a capsule or a tablet. In other implementations, the acid and/or amino acid is/are in a separate tablet/tablets or capsule/capsules than the elemental metal and the source of the nitrate ion. The enhanced activity of co-administration of the nitrate (source of nitrate anion NO₃−) and/or the nitrite (source of nitrite anion NO₂−), the elemental metal, and the amino acid are not reduced if the subject ingests them separately, for example via co-administration of three separate capsules. Accordingly, in some implementations, the composition described herein comprises a capsule including a nitrate (source of nitrate anion NO₃−) and/or the nitrite (source of nitrite anion NO₂−), a capsule comprising an elemental metal, and a capsule comprising an amino acid. In certain implementations of the composition further comprising an acid, the composition can further comprise of a capsule containing the acid.

It is notable that the powdered form of the disclosed composition loses some of its potency when it is administered in water prior to ingestion. Accordingly, administration of the powdered form of the composition should be done while minimizing exposure time to water. Ideally, the exposure of a powdered composition to water should be less than 1 minute, less than 5 minutes, less than 10 minutes, less than 15 minutes, or less than 30 minutes. For example, the subject should ingest the powdered composition and then wash it down with water instead of dissolving the powdered composition in water and drinking the mixture. Or the subject could mix the composition in water and immediately ingest it. Also, if the composition is added in an alkaline mixture (pH above 7), such as water mixed with baking soda, it can retain its effectiveness for at least 10 minutes before ingestion, which is typically enough time for a person to mix his pre-workout drink and consume it.

In some implementations, the composition further comprises a suitable pharmaceutically acceptable coating to prevent moisture from getting in the tablets and/or an additive. Non-limiting examples of the pharmaceutically acceptable coating include waxes, polymers, solid fatty acids, etc. Non-limiting examples of the additive include a carrier, excipient, binder, colorant, flavoring agent, preservative, buffer, diluent, and combinations thereof. In some aspects, the additive is a pharmaceutically acceptable additive or an acceptable food additive.

The present disclosure is also directed to methods of reducing nitrosamine formation in a composition comprising an amine-moiety containing compound and a nitrosating agent. The method comprises adding an elemental metal to the composition comprising the amine-moiety containing compound and the nitrosating agent. In certain implementations, the nitrosating agent is selected from the group consisting of: a nitrate, a nitrite, nitric oxide, and nitrogen dioxide. As shown in the examples, the presence of elemental metal in the composition comprising the amine-moiety containing compound and the nitrosating agent reduces the amount of nitrosamines formed in the composition. In certain implementations, the addition of the elemental metal to the composition can prevent nitrosamine formation. Also as shown in the examples, where the amine-moiety containing compound has undergone nitrosation, the addition of the elemental metal to the composition can undo the reaction. Thus, ingestion of such compositions comprising the amine moiety containing compound, the nitrosating agent, and the elemental metal results in reduced nitrosamine formation, if any is ultimately formed at all.

Accordingly, methods of reducing nitrosamine formation of an amine moiety containing compound are disclosed. The methods comprise co-administering the amine moiety containing compound with an elemental metal and a nitrate and/or a nitrite. In some implementations of the methods disclosed herein, the method further comprises adding an acid to the composition or co-administering the amine moiety containing compound with an elemental metal, a nitrate and/or a nitrite, and an acid. In particular implementations, elemental metals are metallic Magnesium, Iron, Zinc, and Calcium.

### EXAMPLES

### Example 1:

To demonstrate the effectiveness of said compositions and methods in reducing nitrosamine formation in simulated gastric conditions, half a tablespoon of glycine was added in 100 ml of 0.1 N HC1 solution forming a clear solution. 0.1 N HCl is commonly used in the art to simulate the acidic gastric conditions of the stomach. Then ¼ of a tablespoon of sodium nitrite was added while stirring at 200 rpm. The green colored nitrosation product, n-nitrosoglycine, was quickly formed. 1 hour later, ¼ teaspoon of elemental magnesium (granular) was added. 30 minutes later the solution returned to a clear color, indicating elimination of the n-nitrosoglycine.

### Example 2:

To demonstrate the effectiveness of said compositions and methods in reducing nitrosamine formation in simulated gastric conditions, half a tablespoon of 1-tyrosine was added in 100 ml of 0.1 N HCl solution forming a white solution. Then ¼ of a tablespoon of sodium nitrite was added while stirring at 200 rpm. The orange-yellow colored nitrosation product, n-nitrosotyrosine, was quickly formed. 1 hour later, ¼ teaspoon of elemental magnesium (granular) was added. 30 minutes later the solution returned to a white color, indicating elimination of the n-nitrosotyrosine.

### Example 3:

To demonstrate the effectiveness of said compositions and methods in reducing nitrosamine formation in simulated gastric conditions, half a tablespoon of 1-cysteine was added in 100 ml of 0.1 N HCl solution forming a white solution. Then ¼ of a tablespoon of sodium nitrite was added while stirring at 200 rpm. The pink colored S-nitrosation product, S-nitrosocysteine, was quickly formed. 1 hour later, ¼ teaspoon of elemental magnesium (granular) was added. 30 minutes later the solution returned to a white color, indicating elimination of the S-nitrosocysteine.

### Example 4:

In one implementation, the composition includes 1-2 g Amaranthus extract (providing 10-90% nitrate), 50-1000 mg vitamin C, 50-1000 mg magnesium oxide, 10-1000 mg L-cysteine, 50-1000 mg theanine, 5-100 mg elemental iron, 0.5-30 mg folate/5-MTHF, and 1-500 mcg potassium molybdate.

In yet another implementation, the composition is an athletic performance enhancement product (pre-workout product) intended to improve athletic performance indicators such as strength, endurance, vasodilation, and pump (see Table 1). The pre-workout product comprises effective amounts of nitrate (NO₃−) and amino acids. Each serving of the composition provides 5 g of amino acid nitrates, including proline nitrate and beta-alanine nitrate, and the composition is enhanced with hydrogen-reduced elemental iron to further amplify the effects of the nitrates while minimizing nitrosamine formation. Additionally, every nitrate that is in the pre-workout product is provided alongside its regular counterpart (10 g citrulline malate, 2 g betaine anhydrous, and 2.6 g beta-alanine). Other ingredients include a 1 g dose of black tea extract, 200 mg of theanine, and 250 mg of caffeine for energy.

We claim:

1. A composition comprising:
an elemental metal;
a nitrate and/or nitrite; and
an amine moiety containing compound.

2. The composition of claim 1, wherein the elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

3. The composition of claim 1, wherein the amine moiety containing compound is an amino acid.

4. The composition of claim 3, wherein the amino acid is in a salt form.

5. The composition of claim 1, wherein the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine.

6. The composition of claim 1, further comprising an acid.

7. A method of reducing nitrosamine formation from an amine moiety containing compound comprising co-administering the amine moiety containing compound with an elemental metal and a nitrate and/or a nitrite.

8. The method of claim 7, wherein the elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

9. The method of claim 7, wherein the amine moiety containing compound is an amino acid.

10. The method of claim 9, wherein the amino acid is in a salt form.

11. The method of claim 7, wherein the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl-beta-alanine, phenylalanine, proline, serine, threonine, tryptophan, tyrosine, and valine.

12. The method of claim 7, further comprising administering an acid, wherein the acid is co-administered with the amine moiety containing compound, the elemental metal, and the nitrate and/or nitrite.

13. The method of claim 12, wherein nitrosamine formation of the amine moiety containing compound is prevented.

14. A method of reducing nitrosamine formation in a composition comprising an amine moiety containing compound and a nitrosating agent selected from the group consisting of: a nitrate, a nitrite, nitric oxide, and nitrogen dioxide, the method comprising adding an elemental metal to the composition.

15. The method of claim 14, wherein the nitrosating agent is nitric oxide and/or nitrogen dioxide.

16. The method of claim 14, wherein the elemental metal is at least one metal selected from the group consisting of: elemental magnesium, elemental calcium, elemental lithium, elemental zinc, elemental sodium, elemental potassium, elemental beryllium, elemental rubidium, elemental cesium, elemental aluminum, elemental gallium, elemental indium, elemental tin, elemental bismuth, elemental scandium, elemental titanium, elemental vanadium, elemental chromium, elemental manganese, elemental cobalt, elemental manganese, elemental scandium, elemental titanium, nickel, elemental copper, elemental zinc, elemental yttrium, elemental zirconium, elemental niobium, elemental molybdenum, elemental technetium, elemental ruthenium, elemental rhodium, elemental palladium, elemental silver, elemental cadmium, elemental lanthanum, elemental hafnium, elemental tantalum, elemental tungsten, elemental rhenium, elemental osmium, elemental iridium, elemental platinum, elemental gold, and elemental manganese.

17. The method of claim 14, wherein the amine moiety containing compound is an amino acid.

18. The method of claim 16, wherein the amino acid is in a salt form.

19. The method of claim 18, wherein the composition does not form nitrosamines upon ingestion.

20. The method of claim 18, wherein ingestion of the composition comprising the elemental metal forms a lesser amount of nitrosamine compared to ingestion of the composition without the elemental metal.

21. The method of claim 14, wherein the amine moiety containing compound is selected from the group consisting of: agmatine, alanine, beta alanine, arginine, asparagine, aspartic acid, cysteine, glutamine, glutamic acid, glycine, GABA, histidine, isoleucine, leucine, lysine, methionine, phenyl- beta-alanine, phenylalanine, proline, serine, threonine, tryptophan, tyrosine, and valine.

22. The method of claim 14, further comprising adding an acid to the composition.
